SEQUENCE LISTING (10 sequences)

SEQ ID NO: 1 - DNA, 30 bp, HMPV
catataagcatgctatattaaaagagtctc

SEQ ID NO: 2 - DNA, 28 bp, HMPV
cctatttctgcagcatatttgtaatcag

SEQ ID NO: 3 - DNA, 38 bp, HMPV
cgatgcaactgcagtgacaccctcatcattgcacatcg

SEQ ID NO: 4 - DNA, 27 bp, HMPV
tgcaatgatgagggtgtcactgcggtt

SEQ ID NO: 5 - DNA, 30 bp, HMPV; identical to SEQ ID NO: 1

SEQ ID NO: 6 - DNA, 28 bp, HMPV
cctatttctgcagcatatttgtaatcag

SEQ ID NO: 7 - DNA, 59 bp, HMPV
atgcatcgtttttcgatgcatcgatgcaactgcagtgacaccctcatcattgcacatcg

SEQ ID NO: 8 - DNA, 27 bp, HMPV
tgcaatgatgagggtgtcactgcggtt

SEQ ID NO: 9 - DNA, 56 bp, HMPV
gcatcgtttttcgatgctcgatgcaactgcagtgacaccctcatcattgcacatcg

SEQ ID NO: 10 - DNA, 38 bp, HMPV; identical to SEQ ID NO: 3

(19) United States
(12) Patent Application Publication
(10) Pub. No.: US 2011/0104762 A1
(43) Pub. Date: May 5, 2011

(54) Detection probe acting by molecular recognition

(75) Inventors: Eloy Bernal-Mendez, Saint-Quentin-Fallavier (FR); Ali Laayoun, Colombe (FR); Alain Laurent, Grenoble (FR)

(73) Assignee: BIOMERIEUX, Marcy L'Etoile (FR)

(21) Appl. No.: 12/926,962

(22) Filed: Dec. 20, 2010

Related U.S. Application Data
(63) Continuation of application No. 12/225,693, filed on Sep. 26, 2008.

(30) Foreign Application Priority Data
Apr. 25, 2006 (FR) 0651440

(51) Int. Cl. C12P 19/34

(52) U.S. Cl. 435/91.2

(57) ABSTRACT

The present invention relates to a detection probe acting by molecular recognition of a target sequence, comprising successively in the 5′-3′ direction:
a first nucleotide segment comprising a sterically hindering structure at its 5′ end, and
a second nucleotide segment, complementary to the target sequence.

This is a Divisional of application Ser. No. 12/225,693 filed Sep. 26, 2008, which in turn is a National Stage Application of International Patent Application No. PCT/FR2007/051159 filed Apr. 23, 2007. The entire disclosures of the prior applications are hereby incorporated by reference herein in their entirety.

It is often necessary, in technologies relating to nucleic acids and to genetic material, to determine whether a gene, a part of a gene or a nucleotide sequence is present in a living organism, a cell extract of this organism or any other biological, food or industrial sample. There is an enormous value in searching for specific nucleotide sequences, in particular for the detection of pathogenic organisms, the determination of genetic anomalies through the presence of alleles, the detection of the presence of lesions in a host genome and the detection of gene expression through the presence of a particular mRNA or the modification of a cellular host. Many genetic diseases can thus be diagnosed by analyzing and even quantifying the expression of certain genes.

Various types of nucleic acid-specific detection methods are described in the literature. These methods are generally based on the pairing properties of the complementary nucleic acid strands, commonly termed “nucleic acid hybridization” or simply “hybridization”.

In general, after having determined the specific sequence of an organism or of a disease which should be analyzed, it is advisable to extract the nucleic acids from a sample and optionally to amplify and detect the sequence of interest. Many amplification and detection methods have been developed for this purpose. Thus, PCR (polymerase chain reaction) is based on the repetition of a three-step process: denaturation of the double-stranded DNA so as to obtain two separate complementary single strands, hybridization of each primer to a strand of single-stranded DNA, and enzymatic extension of the primers via a thermostable DNA polymerase, for example Taq DNA polymerase which synthesizes a DNA strand, or amplicon, having a sequence complementary to that acting as target. The amplification can be analyzed at the end of cycles (end-point PCR), or followed in real time (real-time PCR) through the detection of the amplicons by fluorescence: the amplification and the detection of the amplicons may therefore be sequential or simultaneous. Several real-time PCR techniques exist. One of them uses particular hairpin probes called “molecular beacons”, comprising a “loop” part, complementary to the target amplicon to be analyzed, and two “stem” parts, complementary to one another. The “molecular beacons” are nucleotide sequences comprising a fluorophore at one end of one of the “stem” parts and a fluorescence “quencher” at the end of the other “stem” part. In the absence of complementary target amplicon, these “molecular beacons” adopt a “hairpin” configuration: the 5′ and 3′ ends are close by virtue of the hybridization of the two “stem” parts; there is no emission of fluorescence. In the presence of amplicon, the “loop” part hybridizes to the amplicon, thereby causing distancing of the two “stem” parts, the “quencher” no longer plays its role and there is emission of fluorescence.

However, during a PCR, the distancing of the fluorophore and of the “quencher”, which allows the emission of a detectable signal, may be due not to the hybridization of the probe to the amplicon, but to a degradation of the probe due to the thermostable polymerase enzyme used during the amplification reaction. This is because thermostable polymerase enzymes, and in particular the Taq polymerase enzyme, have a 5′ nuclease activity. As a result, the signal emitted at each cycle is no longer directly proportional to the amount of amplicons produced (signal generated by molecular recognition between the “molecular beacon” and the amplicon), but will also depend on the cleavage due to the thermostable polymerase enzyme. The signal is therefore no longer quantitative. Furthermore, in addition to the “parasitic” fluorescence which appears during a real-time PCR, the cleavage of the “molecular beacons” due to the residual presence of thermostable polymerase enzyme at the cycle end point affects a post-amplification temperature gradient analysis. However, this post-amplification temperature gradient analysis is the most sensitive method for detecting simple sequence variations, such as SNPs (single nucleotide polymorphisms) by the “molecular beacons”.

It is therefore very important to increase the sensitivity of the PCR technique, by preventing any parasitic cleavage by the thermostable polymerase enzymes used during a PCR, in particular the Taq polymerase enzyme.

The present invention intends to solve all the drawbacks of the prior art by improving the sensitivity and the reliability of detection by real-time or end-point PCR. In this respect, the invention relates to new labeled oligonucleotides resistant to the 5′ nuclease activity of thermostable polymerase enzymes such as Taq polymerase.

To this effect, the present invention relates to a detection probe acting by molecular recognition of a target sequence, comprising successively in the 5′-3′ direction:
a first nucleotide segment comprising a sterically hindering structure at its 5′ end, and
a second nucleotide segment, complementary to the target sequence.

According to a preferred embodiment of the invention, the sterically hindering structure is an arborescence, hairpin or double-stranded structure.

According to a preferred embodiment of the invention, the detection probe acting by molecular recognition also comprises, downstream of said second nucleotide segment, a third nucleotide segment capable of hybridizing to said first nucleotide segment.

According to a preferred embodiment of the invention, the detection probe acting by molecular recognition is labeled at one end with a fluorophore and at the other end with a quencher.

Preferably, said third segment is completely or partially complementary to said first segment such that, under favorable temperature and salinity conditions, in particular the conditions of the hybridization and polymerization steps in a PCR reaction, these two complementary parts interact with one another, forming an intramolecular double helix.

According to a preferred embodiment of the invention, said first segment comprises a fluorophore and said third segment comprises a quencher. According to another preferred embodiment of the invention, said first segment comprises a quencher and said third segment comprises a fluorophore.

According to a preferred embodiment of the invention, said first and/or third segments preferably comprise from 3 to 8 nucleosides, even more preferably from 4 to 6 nucleosides.

According to a preferred embodiment of the invention, said second segment preferably comprises from 10 to 35 nucleosides and even more preferably from 15 to 25 nucleosides.

The following definitions will make it possible to understand the invention more clearly.

For the purpose of the present invention, the term “upstream” is intended to mean a region located on the 5′-end side of the nucleic acid or of the polynucleotide sequence, and the term “downstream” is intended to mean a region located on the 3′-end side of said nucleic acid or of said polynucleotide region.

For the purpose of the present invention, the terms nucleotide fragment, nucleic acid fragments, nucleotide segment, nucleic acid segment, nucleotide sequence, nucleic acid sequence, or oligonucleotide denote a natural DNA or RNA fragment, a natural or synthetic polynucleotide, or a synthetic DNA or RNA fragment which is unmodified or which comprises at least one modification, whether on the nucleobase (modified base such as inosine, methyl-5-deoxycitidine, dimethylamino-5-deoxyuridine, deoxyuridine, diamino-2,6-purine, bromo-5-deoxyuridine, pseudouridine, pseudoisocytidine or any other modified base), on the sugar (such as 2′-OMe, locked nucleic acids, alpha-nucleotides or the like) or on the phosphate (phosphorothioate, phosphoramidate and the like). Each of the modifications may be taken in combination.

The expression detection probe acting by molecular recognition is intended to mean a nucleic sequence of from 10 to 100 nucleotide units, in particular from 15 to 45 nucleotide units, having a hybridization specificity under given conditions so as to form a hybridization complex with a target nucleic sequence and emitting a signal when the probe hybridizes to the target nucleic sequence.

The detection probe may in particular be a “molecular beacon” detection probe as described by Tyagi & Kramer (Nature Biotech, 1996, 14:303-308). These “molecular beacons” become fluorescent at the time of the hybridization. They have a stem-loop structure and contain a fluorophore and a “quencher” group. The binding of the specific loop sequence with its complementary target nucleic acid sequence brings about uncoiling of the stem and the emission of a fluorescent signal during excitation at the appropriate wavelength.

The detection probe may in particular be a “Scorpion®” or “Amplifluor®” detection probe. The Scorpions® are sequences which have, on the 5′ side, a sequence with a stem-loop structure with a “quencher” at one end of said structure and a fluorophore at the other end of said structure. On the 3′ side, the Scorpions® have a sequence which is complementary to a sequence of the target and which acts as a primer during the amplification reaction. Between the primer sequence and the stem-loop sequence, a “spacer” makes it possible to prevent recognition of the stem-loop sequence by the polymerase. A “spacer” is a synthetic molecule which is incorporated, into a nucleotide sequence during synthesis so as to separate said sequence into two parts, in order to produce different effects according to its nature.

The Amplifluors® are nucleotide sequences which have, on the 5′ side, a sequence with a stem-loop structure with a fluorophore at the 5′ end and a “quencher” of Dabsyl type at the other end of the structure. On the 3′ side, the Amplifuors® have a sequence which is complementary to a sequence of the target and which acts as a primer during the amplification reaction.

For the purpose of the present invention, the term “fluorophore” is intended to mean a molecule which emits a fluorescence signal between 500 and 700 nm when it is excited by light at a suitable wavelength (or between 450 and 650 nm). The fluorophore may in particular be a rhodamine or a derivative such as Texas Red, a fluorescein or a derivative, such as 5-bromomethylfluorescein, a fluorophore of the Alexa family such as Alexa532, Alexa647, Alexa 405, Alexa 700 or Alexa 680, or any other fluorophore which is suitable according to the measuring apparatus used. The available fluorophores for the detection probes are very varied and known to those skilled in the art.

For the purpose of the present invention, the term “fluorescein” is intended to mean an aromatic chemical molecule which emits a fluorescence signal with a maximum emission around 530 nm, when it is excited by light at a wavelength of around 495 nm.

For the purpose of the present invention, the term “quencher” is intended to mean a molecule which interferes with the fluorescence emitted by a fluorophore. This quencher is in particular chosen from nonfluorescent aromatic molecules, so as to avoid parasitic emissions. Preferably, said “quencher” is a Dabsyl or a Dabcyl or a “Black hole Quencher™”. Dabcyl, Dabsyl and the “Black hole Quenchers™” are nonfluorescent aromatic molecules which prevent the emission of fluorescence when they are physically in proximity to a fluorophore, or by FRET.

For the purpose of the present invention, the expression “nucleotide segment capable of hybridizing to a target sequence” is intended to mean a sequence or a region which can hybridize to another sequence/region under hybridization conditions, which can be determined in each case in a known manner. The term complementary sequences/regions is also used. A sequence or a region which is strictly complementary to another is a sequence in which each of the bases can pair with a base of the other sequence, without mismatching.

The term hybridization is intended to mean the process during which, under appropriate conditions, two nucleotide fragments having sufficiently complementary sequences are capable of forming a double strand with stable and specific hydrogen bonds. The hybridization conditions are determined by the stringency, i.e. the strictness and the low salinity of the operating conditions. The higher the stringency at which the hybridization is carried out, the more specific it is. The stringency is defined in particular according to the base composition of a probe/target duplex, and also by the degree of mismatching between two nucleic acids. The stringency may also depend on the reaction parameters, such as the concentration and the type of ionic species present in the hybridization solution, the nature and the concentration of denaturing agents and/or the hybridization temperature. The stringency of the conditions under which a hybridization reaction should be carried out will depend mainly on the hybridization probes used. All these data are well known and the appropriate conditions can be determined by those skilled in the art.

For the purpose of the present invention, the term “sterically hindering structure” is intended to mean a structure, in particular nucleotide structure, which imposes a steric stress capable of blocking the 5′-nuclease activity of a thermostable polymerase enzyme such as a DNA polymerase enzyme, responsible for the degradation of an oligonucleotide, i.e. a structure having a size greater than that of a single-stranded DNA. Preferably, the sterically hindering structure has at least the size of a DNA double helix, i.e. a minimum diameter of at least 20 angstroms.

According to a preferred embodiment of the invention, the sterically hindering structure is an arborescence structure. This makes it possible to increase the size of the hindering structure without considerably increasing the number of cycles for synthesizing the probe. This also makes it possible to modulate the size of the structure as appropriate. The size of such a structure is equal to or greater than that of a double helix.

For the purpose of the present invention, the term “arborescence structure” is intended to mean a sequence in which a nucleotide is coupled via its 5′ side to another nucleotide, or any other molecule, in particular molecule which can be 5′-coupled during the synthesis of a nucleotide, and which is modified and which has two or more 5′ ends, and which as a result can be coupled with two or more other nucleotides in parallel. If these two or more last nucleotides are also modified in the same manner, there will then be four 5′ ends which can be coupled to four nucleotides in parallel, and so on. The term “arborescence structure” is used, as opposed to the linear structure of natural nucleic acids.

According to another preferred embodiment of the invention, the sterically hindering structure is a hairpin structure. The advantage of such a structure is that it can be produced without having to resort to reagents other than those used for the normal synthesis of DNA nucleotide probes and without modifying the synthesis cycles. This structure can also be obtained by linear synthesis. It is practical from the point of view of the characterization and quality control because the same analytical techniques as for conventional oligonucleotides and probes can be used. Furthermore, due to the ease with which it is synthesized and its simplicity, it is easy for those skilled in the art to modulate this structure according to needs in terms of nucleotide sequence and length. The sequence length required for the formation of such a structure is that which enables it to remain stable under the salinity conditions and at the working temperature of the DNA polymerase during the polymerization reaction in the PCR. The size of the structure, in the hindrance sense, is that of a double helix.

The term hairpin structure is intended to mean a natural or modified, linear nucleotide sequence in which a part of the sequence is complementary to another part of the sequence, in such a way that, under favorable temperature and salinity conditions, in particular the conditions of the hybridization and polymerization steps in a PCR reaction, these two complementary parts interact with one another, forming an intramolecular double helix.

According to another preferred embodiment of the invention, the sterically hindering structure is a double-stranded structure. The advantage of such a structure is that it can be produced without having to resort to reagents other than those used for the normal synthesis of DNA nucleotide probes and without modifying the synthesis cycles. The term double-stranded structure is intended to mean two complementary nucleotide sequences which hybridize to one another so as to form a double helix under favorable temperature and salinity conditions, in particular the conditions of the hybridization and polymerization steps in a PCR reaction. Compared with the hairpin structure, a double-stranded structure makes it possible to have fewer nucleotides to be added in 5′ of the nucleic probe, thereby improving the synthesis and purification yield. The second strand which forms the double strand is another molecule which can be synthesized separately.

The invention also relates to the use of a sterically hindering structure for blocking a 5′ nuclease activity of a polymerase enzyme.

The invention also relates to the use of at least one detection probe acting by molecular recognition as defined above and which blocks the 5′ nuclease activity of a thermostable polymerase enzyme during a polymerase chain reaction.

Such uses are highly relevant since it is thus possible to detect a target sequence by using probes of “molecular beacon” type while preventing the cleavage thereof by the 5′ nuclease activity of a polymerase enzyme during real-time polymerase chain reaction or during post-amplification end-point measurements.

The invention also relates to a method for detecting a nucleic material in a biological sample, comprising the following steps:
a) the nucleic material of a biological sample is extracted,
b) the nucleic material is amplified so as to obtain amplicons of at least one target sequence of the nucleic material in the presence of a polymerase enzyme,
c) at least one detection probe acting by molecular recognition as described above is used, simultaneously with amplification step b) or after amplification step b),
d) the presence of said amplicons is detected.

For the purpose of the present invention, the term “nucleic material” is intended to mean a nucleic acid sequence such as a deoxyribonucleic acid (DNA) or ribonucleic acid (RNA) sequence. According to a preferred embodiment of the invention, the nucleic material comprises a deoxyribonucleic acid sequence. According to a preferred embodiment of the invention, the nucleic material is extracted from a biological sample taken from a patient.

For the purpose of the present invention, the term “biological sample” is intended to mean any sample that may contain a nucleic material as defined hereinafter. This biological sample may be taken from a patient and may in particular be a tissue sample, blood sample, serum sample, saliva sample or circulating cell sample from the patient. This sample may also be a food sample. This biological sample is obtained by taking it in any way known to those skilled in the art.

For the purpose of the present invention, the term “target sequence” is intended to mean a nucleotide sequence of which at least a part of the series of nucleotide units is specific and complementary to the nucleotide sequence of the detection probe used.

For the purpose of the present invention the term “thermostable polymerase enzyme” is intended to mean a natural or modified enzyme which is capable of synthesizing a DNA strand of sequence complementary to that present in the reaction medium, which is used as target or model, starting from the 3′ side of a primer of sequence complementary to the target, and using nucleotide triphosphates as substrate, and capable of withstanding, without loss of its biological activity, high temperatures, in particular up to 95° C. for a few minutes.

For the purpose of the present invention, during step a) the nucleic material of a biological sample is extracted by any protocol known to those skilled in the art. By way of indication, the nucleic acid extraction can be carried out by means of a step of lysis of the cells present in the biological sample, in order to release the nucleic acids contained in the protein and/or lipid envelopes of the cells (such as cell debris which disturbs the subsequent reactions). By way of example, use may be made of the lysis methods as described in patent applications WO 00/05338 on mixed magnetic and mechanical lysis, WO 99/53304 on electrical lysis, and WO 99/15321 on mechanical lysis.

Those skilled in the art may use other well-known methods of lysis, such as heat shock or osmotic shock, or chemical lysis with chaotropic agents such as guanidium salts (U.S. Pat. No. 5,234,809). This lysis step may also be followed by a purification step, allowing the nucleic acids to be separated from the other cellular constituents released in the lysis step. This step generally makes it possible to concentrate the nucleic acids, and can be adapted to the purification of DNA or of RNA. By way of example, use may be made of magnetic particles optionally coated with oligonucleotides, by adsorption or covalence (in this respect see patents U.S. Pat. No. 4,672,040 and U.S. Pat. No. 5,750,338), and the nucleic acids which have bound to these magnetic particles can thus be purified by means of a washing step. This nucleic acid purification step is particularly advantageous if it is desired to subsequently amplify said nucleic acids. A particularly advantageous embodiment of these magnetic particles is described in patent applications WO 97/45202 and WO 99/35500. Another advantageous example of a nucleic acid purification method is the use of silica, either in the form of a column or in the form of inert particles (Boom R. et al., J. Clin. Microbiol., 1990, No. 28(3), p. 495-503) or magnetic particles (Merck: MagPrep® Silica, Promega: MagneSil™ Paramagnetic particles). Other very widely used methods are based on ion exchange resins in a column or in a paramagnetic particulate format (Whatman: DEAE-Agarose) (Levison P R et al., J. Chromatography, 1998, p. 337-344). Another highly relevant but not exclusive method for the invention is that of adsorption onto a metal oxide support (Xtrana company: Xtra-Bind™ matrix).

When it is desired to specifically extract the DNA from a biological sample, it is in particular possible to carry out an extraction with phenol, chloroform and alcohol so as to eliminate the proteins, and to precipitate the DNA with 70% alcohol. The DNA can then be pelleted by centrifugation, washed and resuspended.

For the purpose of the present invention, “step b)” is a process which generates multiple copies (or amplicons) of a nucleic sequence through the action of at least one polymerase enzyme. For the purpose of the present invention, the term amplicons is intended to mean the copies of the target sequence that are obtained during an enzyme amplification reaction. Such amplification reactions are well known to those skilled in the art and mention may in particular be made of PCR (polymerase chain reaction), as described in patents U.S. Pat. No. 4,683,195, U.S. Pat. No. 4,683,202 and U.S. Pat. No. 4,800,159.

In general, step b) is a succession of cycles comprising the following steps:
the denaturation of the target sequence in order to obtain two complementary DNA target strands, or to destructure the RNA target strand,
the hybridization of each of the target strands, obtained during the preceding denaturation step, with at least one amplification primer,
the formation, from the amplification primers, of the strands complementary to the strands to which they have hybridized in the presence of a polymerase enzyme and of nucleoside triphosphates,
   this cycle being repeated a given number of times so as to obtain the target sequence at a sufficient concentration to allow its detection.

Steps b) and c) are carried out at the same time or one after the other.

When steps b) and c) are carried out at the same time, this embodiment is preferably implemented by “real-time PCR”, which combines in a single step the PCR amplification technique and the detection, and which makes use in particular of “molecular beacons”. The PCR reaction takes place in the tube, producing amplicons with which the specific “molecular beacons” can hybridize so as to give a fluorescent signal. The formation of the new DNA molecules is measured in real time by verifying the signal in a fluorescent reader, during the hybridization step. The use of labeled oligonucleotides according to the present invention makes it possible to prevent the detection probes from being degraded by the amplification enzyme (for example, Taq polymerase), thereby increasing the sensitivity of the detection and improving the effectiveness of the technique.

When steps b) and c) are carried out one after the other, the PCR reaction occurs in the tube, producing amplicons. At the end of this amplification step, the “molecular beacons” are added to the reaction medium, and can hybridize so as to give a fluorescent signal. The use of labeled oligonucleotides according to the present invention makes it possible to prevent the detection probes from being degraded by the thermostable polymerase amplification enzyme, which remains residually in the reaction tube, thereby increasing the sensitivity of the detection and improving the effectiveness of the technique.

“Step d)” is carried out by detecting the fluorescence signal emitted at the time of the hybridization of the labeled oligonucleotide according to the invention to the amplicon, and can be carried out by any of the protocols known to those skilled in the art.

The figures attached hereto are given by way of explanatory example and are in no way limiting in nature. They will make it possible to understand the invention more clearly.

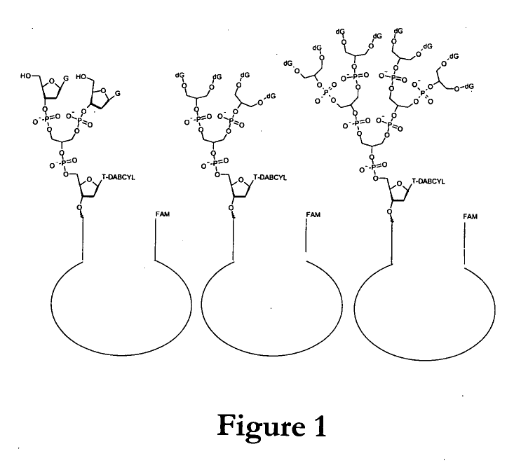

FIG. 1 shows “molecular beacons” according to the invention, comprising a sterically hindering arborescence structure.

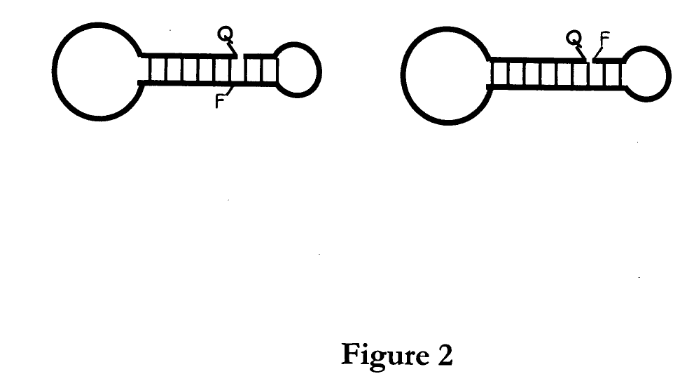

FIG. 2 shows “molecular beacons” according to the invention, comprising, on the 5′ side, a sterically hindering hairpin structure. The continuous line represents the nucleotide sequence. The letter “Q” represents the “quencher” located at the 3′ end. The letter “F” represents the fluorophore located on a T base inside the sequence, in the first segment of the probe (left-hand drawing) and the fluorophore located at the 5′ end of the sequence, in the hairpin structure (right-hand drawing). The vertical lines represent the hybridization, in the segments which form double helices.

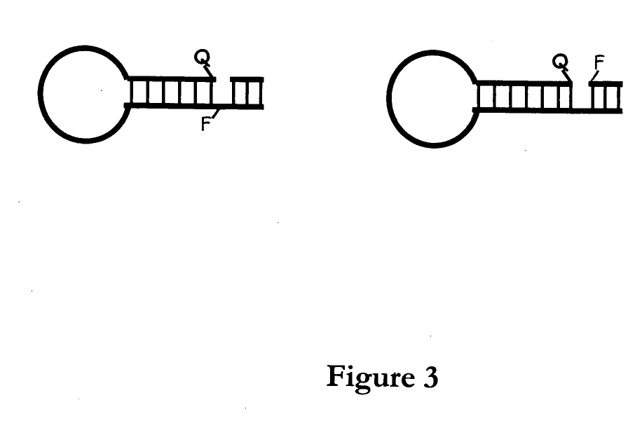

FIG. 3 shows “molecular beacons” according to the invention, comprising, on the 5′ side, a sterically hindering double-stranded structure. The continuous lines represent the nucleotide sequences. The letter “Q” represents the “quencher” located at the 3′ end of the sequence of the “molecular beacons”. The letter “F” represents the fluorophore located on a T base inside the sequence of the “molecular beacon”, in the first segment of the probe (left-hand drawing) and the fluorophore located at the 5′ end of the sequence of the complementary oligonucleotide which serves to form the sterically hindering double-stranded structure (right-hand drawing). The vertical lines represent the hybridization, in the segments which form double helices.

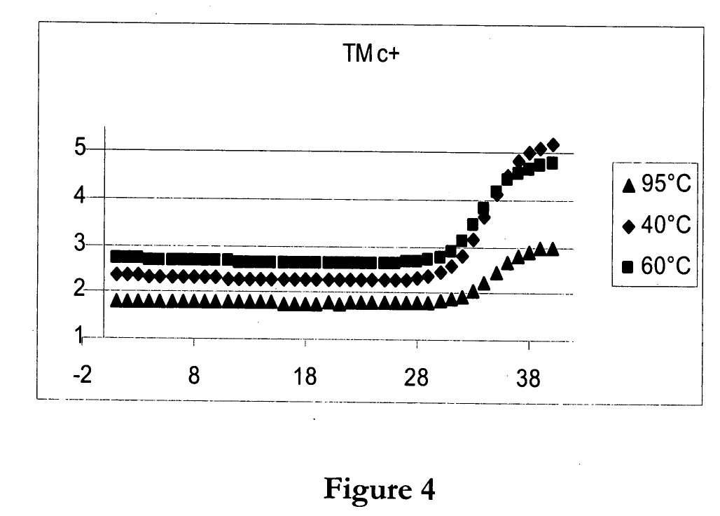

FIG. 4 shows the fluorescence profiles measured at 40, 60 and 95° C., for the TaqMan probe, as described in example 1.

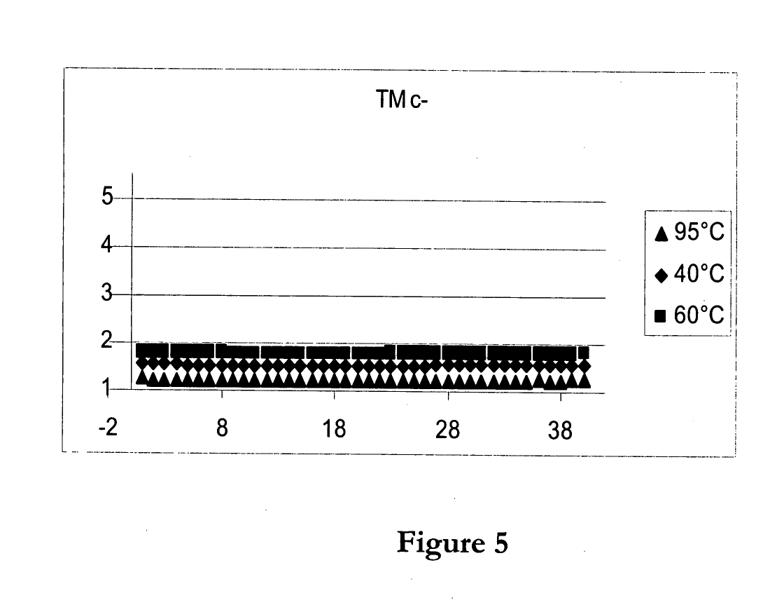

FIG. 5 shows the fluorescence profiles measured at 40, 60 and 95° C., for the negative control for the TaqMan probe, as described in example 1.

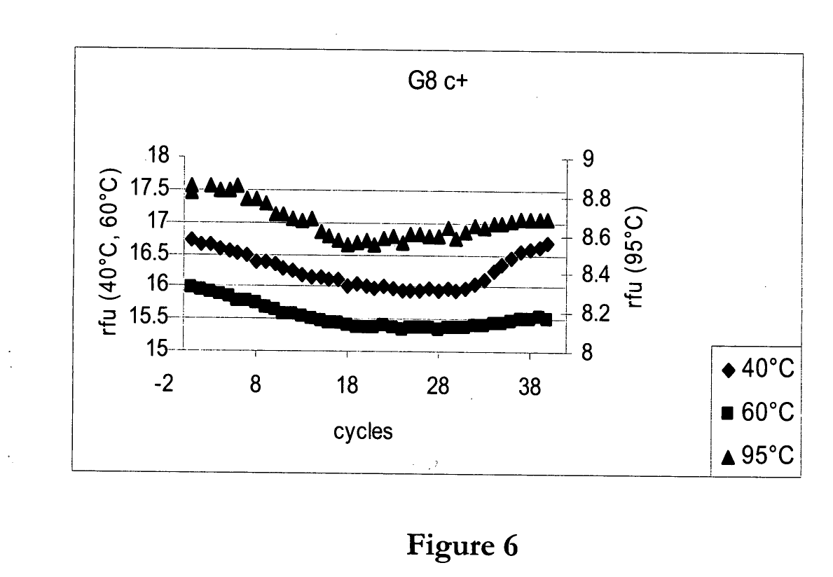

FIG. 6 shows the fluorescence profiles measured at 40, 60 and 95° C., for the G8 “molecular beacon” as described in example 1.

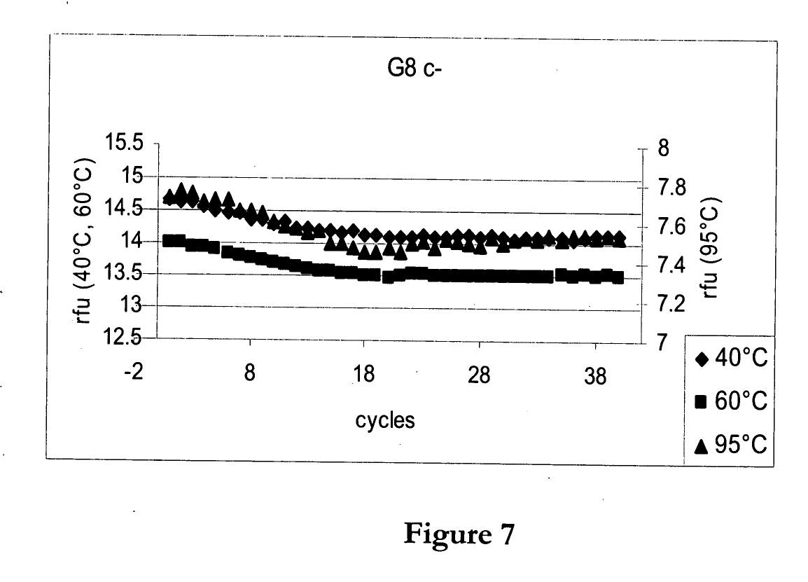

FIG. 7 shows the fluorescence profiles measured at 40, 60 and 95° C., for the negative control for the G8 “molecular beacon”, as described in example 1.

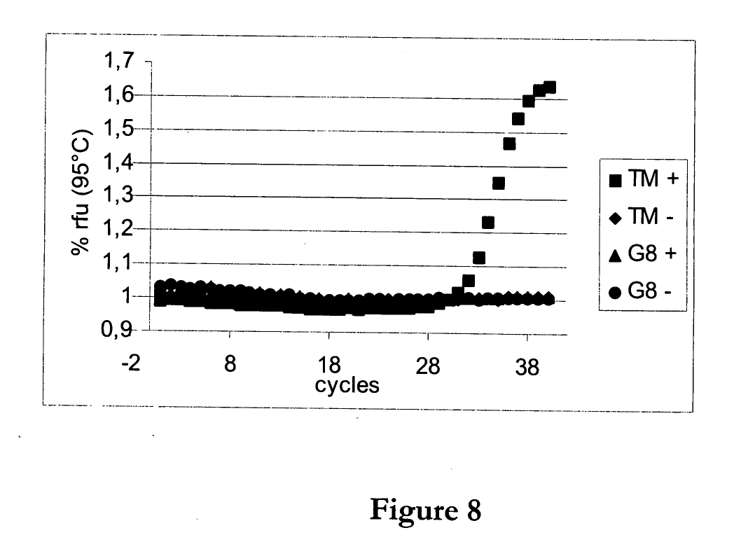

FIG. 8 shows the normalized fluorescence profiles measured at 95° C., for the G8 “molecular beacon” and for the TaqMan probe TM during the amplification on a Lightcycler, as described in example 1.

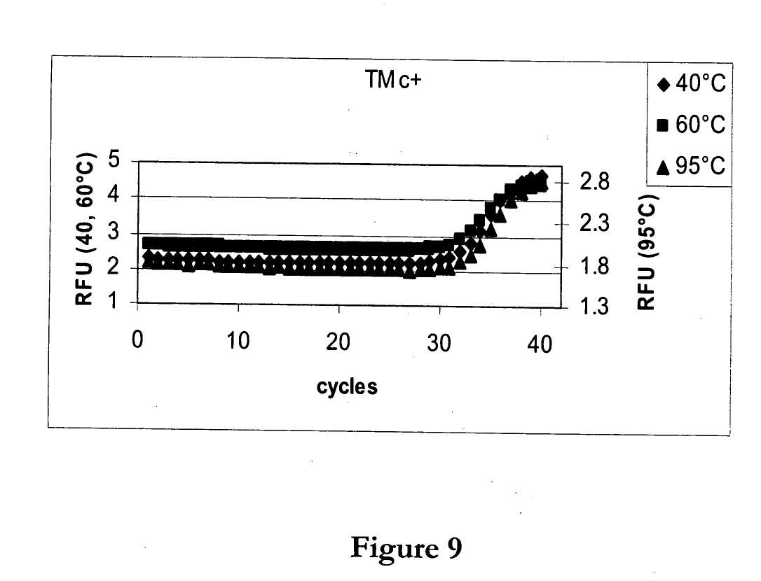

FIG. 9 shows the fluorescence profiles measured at 40, 60 and 95° C., for the TaqMan probe, as described in example 2.

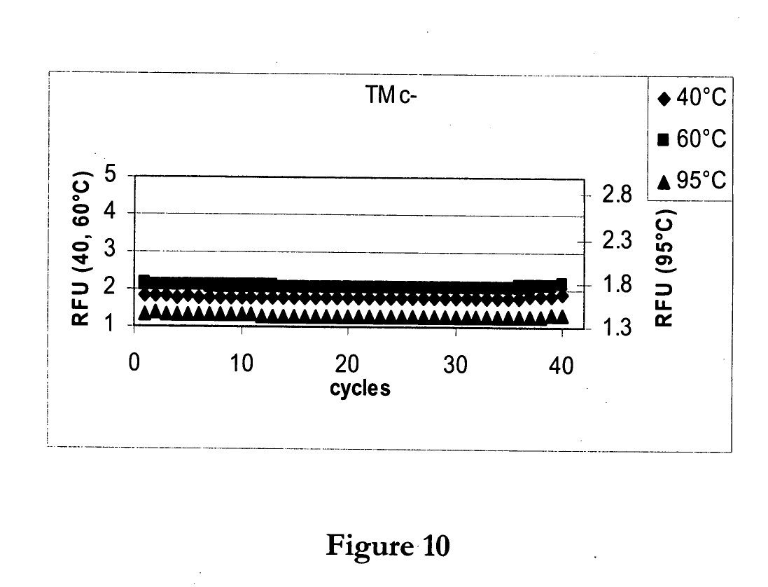

FIG. 10 shows the fluorescence profiles measured at 40, 60 and 95° C., for the negative control for the TaqMan probe, as described in example 2.

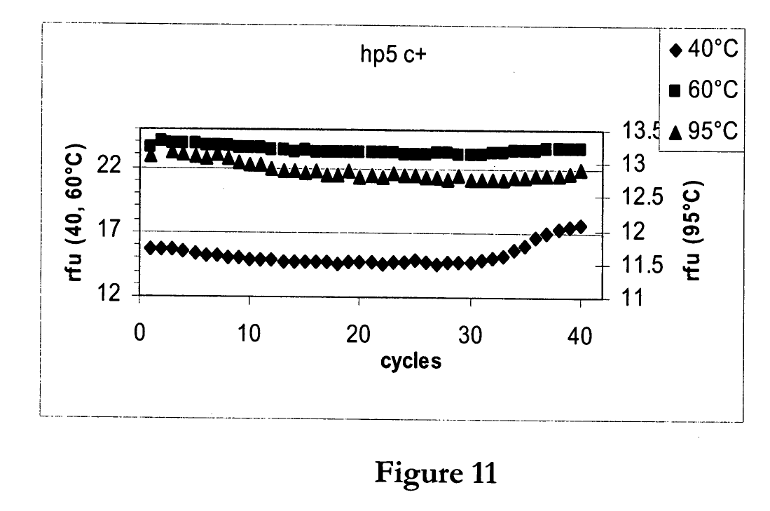

FIG. 11 shows the fluorescence profiles measured at 40, 60 and 95° C., for the hp5 “molecular beacon”, as described in example 2.

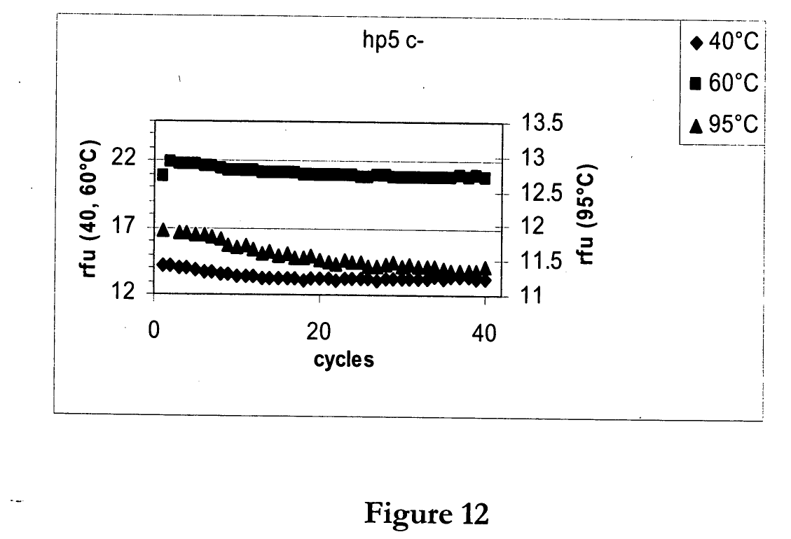

FIG. 12 shows the fluorescence profiles measured at 40, 60 and 95° C., for the negative control for the hp5 “molecular beacon”, as described in example 2.

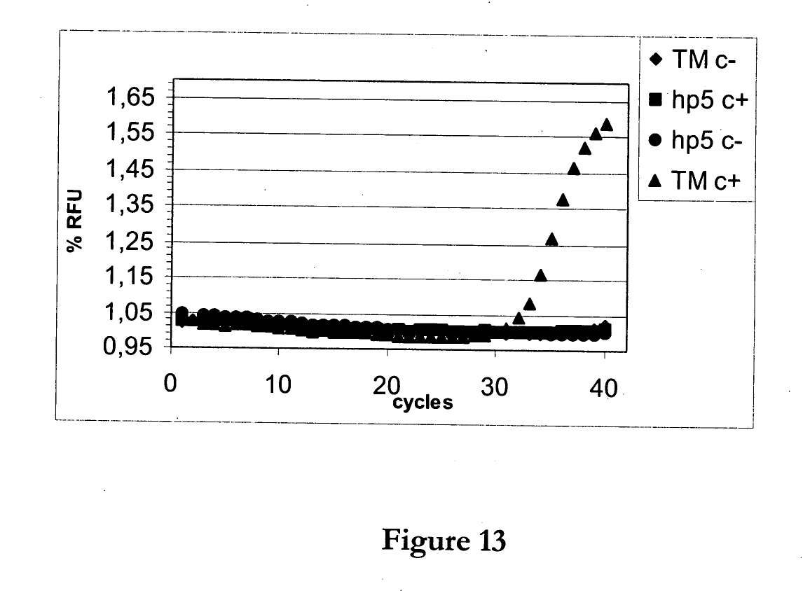

FIG. 13 shows the normalized fluorescence profiles measured at 95° C., for the hp5 “molecular beacon” and the TaqMan probe TM during the amplification on a Lightcycler, as described in example 2.

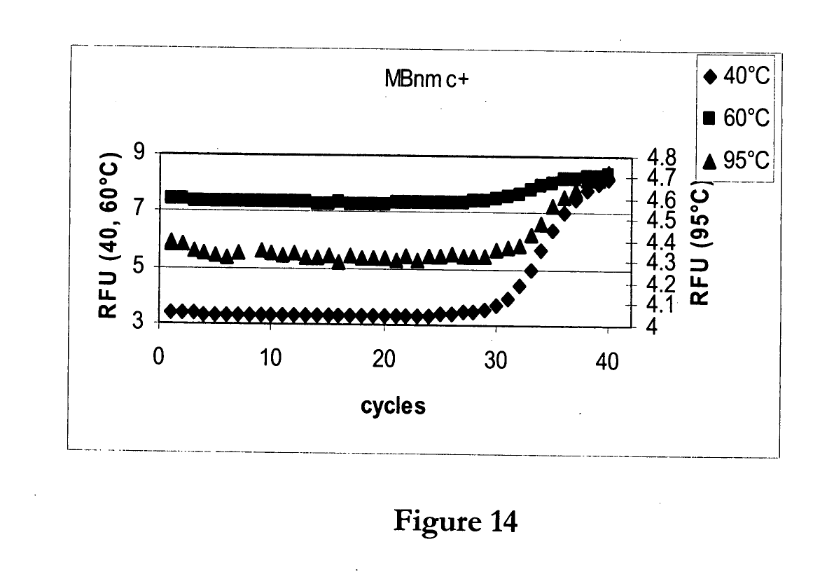

FIG. 14 shows the fluorescence profiles measured at 40, 60 and 95° C., for the control “molecular beacon” as described in example 3.

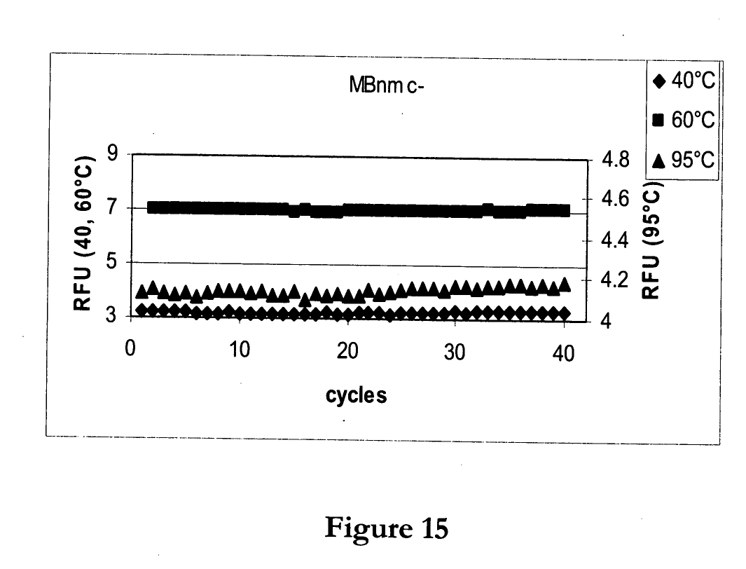

FIG. 15 shows the fluorescence profiles measured at 40, 60 and 95° C., for the negative control for the control “molecular beacon”, as described in example 3.

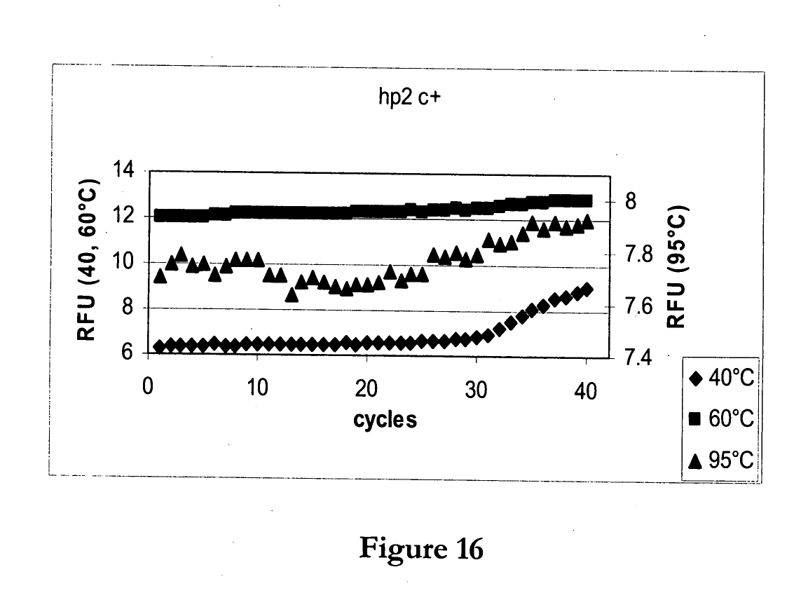

FIG. 16 shows the fluorescence profiles measured at 40, 60 and 95° C., for the hp2 “molecular beacon”, as described in example 3.

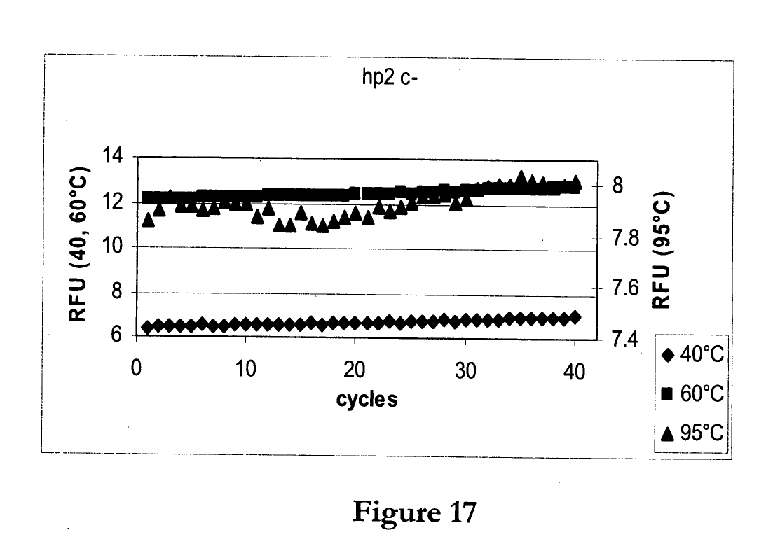

FIG. 17 shows the fluorescence profiles measured at 40, 60 and 95° C., for the negative control for the hp2 “molecular beacon”, as described in example 3.

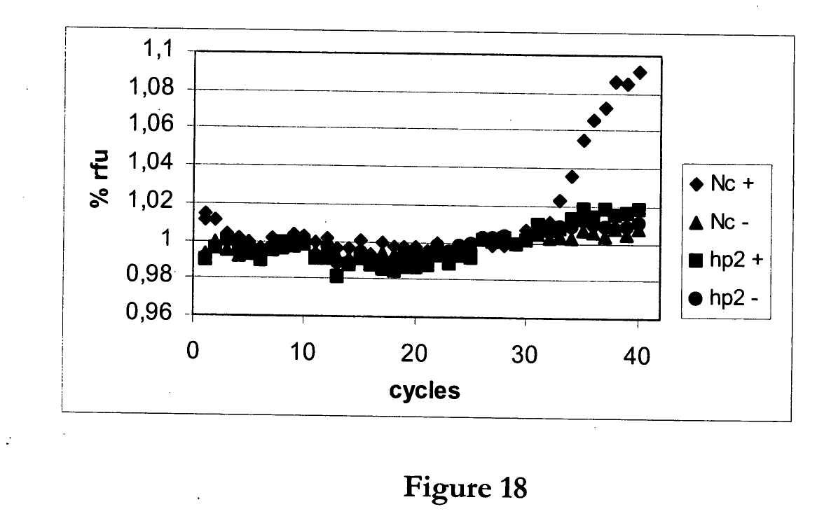

FIG. 18 shows the normalized fluorescence profiles measured at 95° C., for the hp2 “molecular beacon” and the MBnm “molecular beacon” during the amplification on a Lightcycler, as described in example 3.

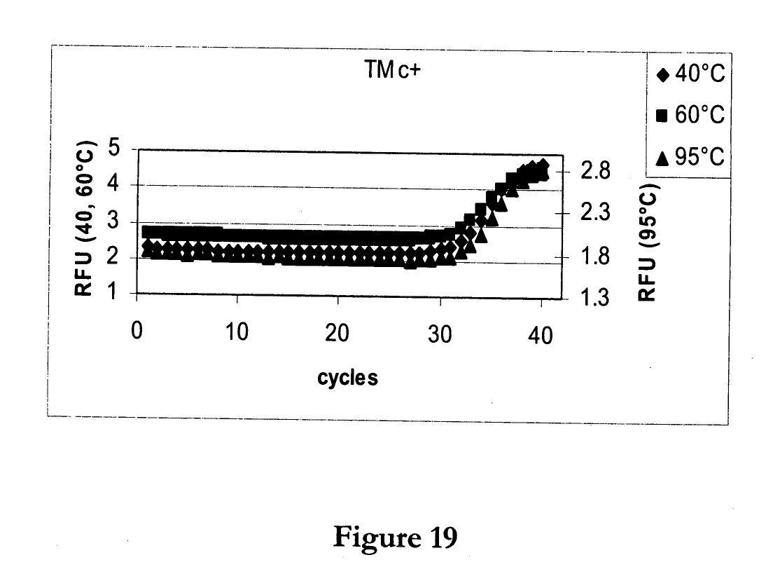

FIG. 19 shows the fluorescence profiles measured at 40, 60 and 95° C., for the TaqMan probe, as described in example 4.

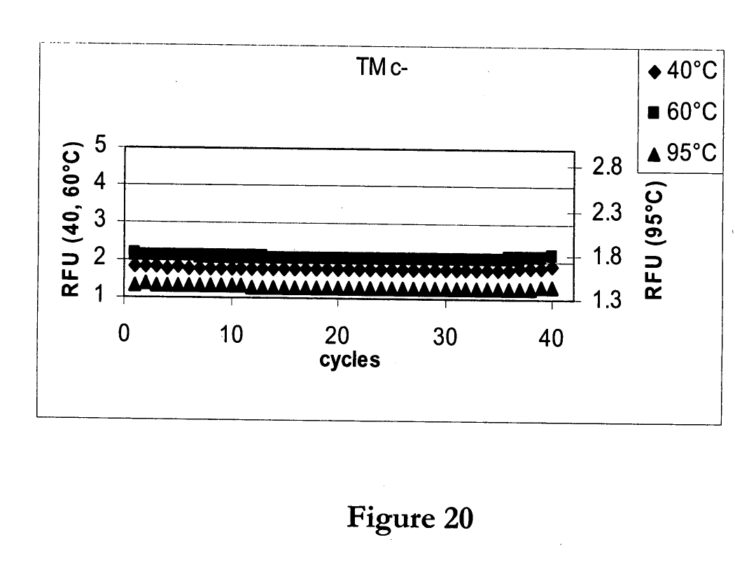

FIG. 20 shows the fluorescence profiles measured at 40, 60 and 95° C., for the negative control for the TaqMan probe, as described in example 4.

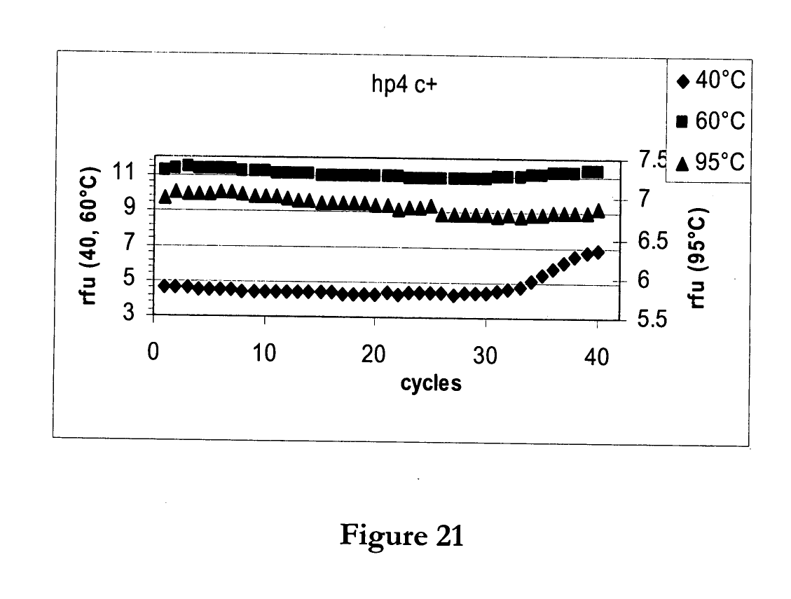

FIG. 21 shows the fluorescence profiles measured at 40, 60 and 95° C., for the hp4 “molecular beacon”, as described in example 4.

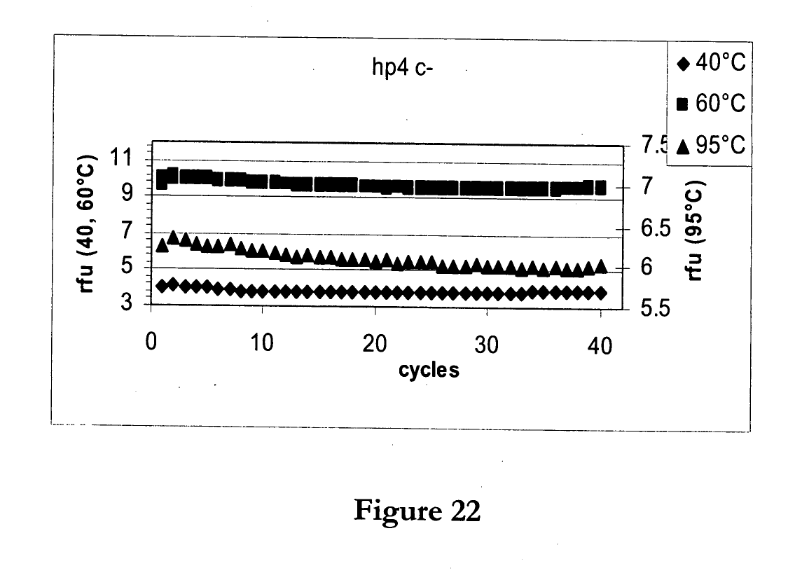

FIG. 22 shows the fluorescence profiles measured at 40, 60 and 95° C., for the negative control for the hp4 “molecular beacon”, as described in example 4.

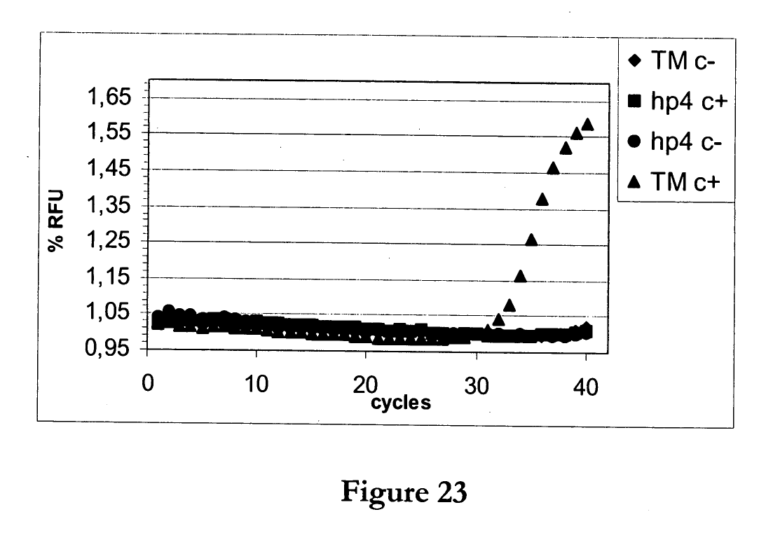

FIG. 23 shows the normalized fluorescence profiles measured at 95° C., for the hp4 “molecular beacon” and the TaqMan probe TM during the amplification on a Lightcycler, as described in example 4.

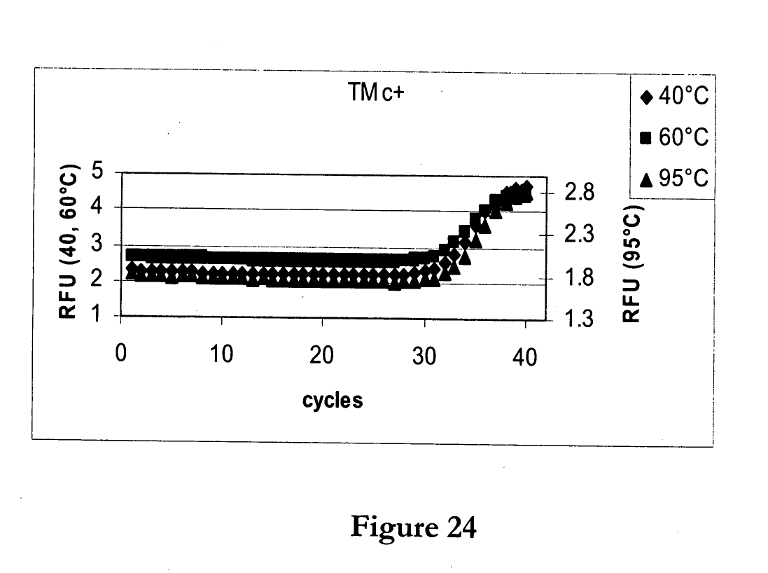

FIG. 24 shows the fluorescence profiles measured at 40, 60 and 95° C., for the TaqMan probe, as described in example 5.

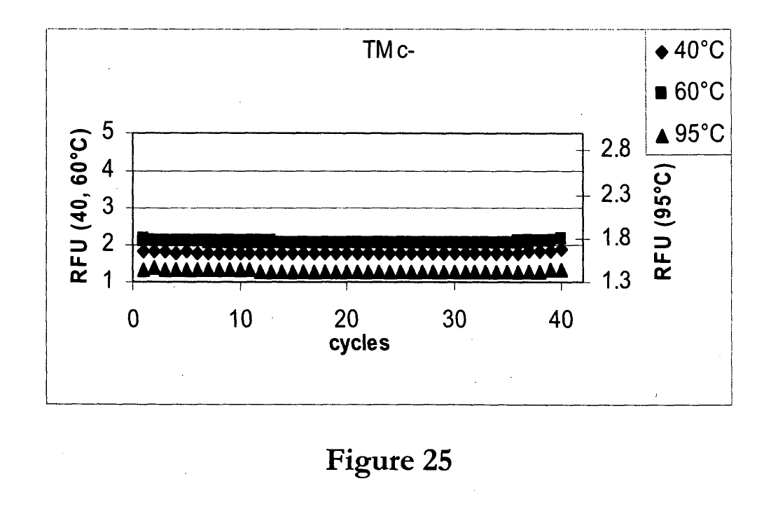

FIG. 25 shows the fluorescence profiles measured at 40, 60 and 95° C., for the negative control for the TaqMan probe, as described in example 5.

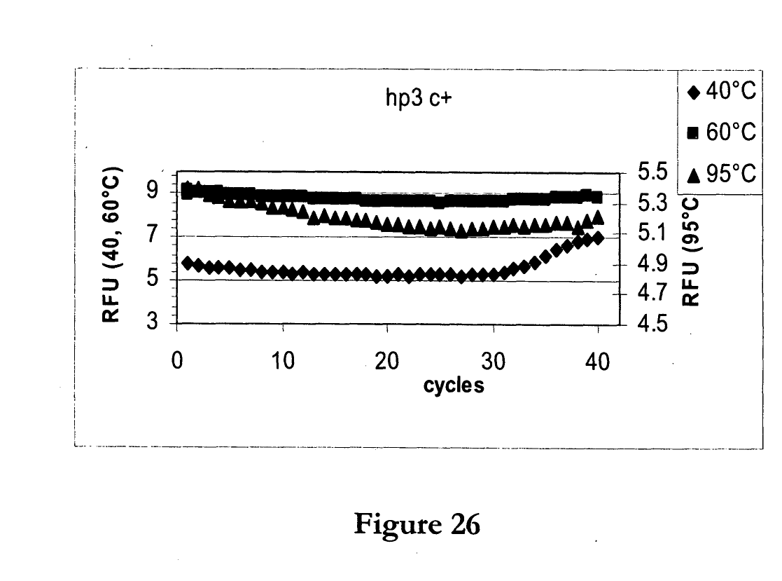

FIG. 26 shows the fluorescence profiles measured at 40, 60 and 95° C., for the hp3 “molecular beacon”, as described in example 5.

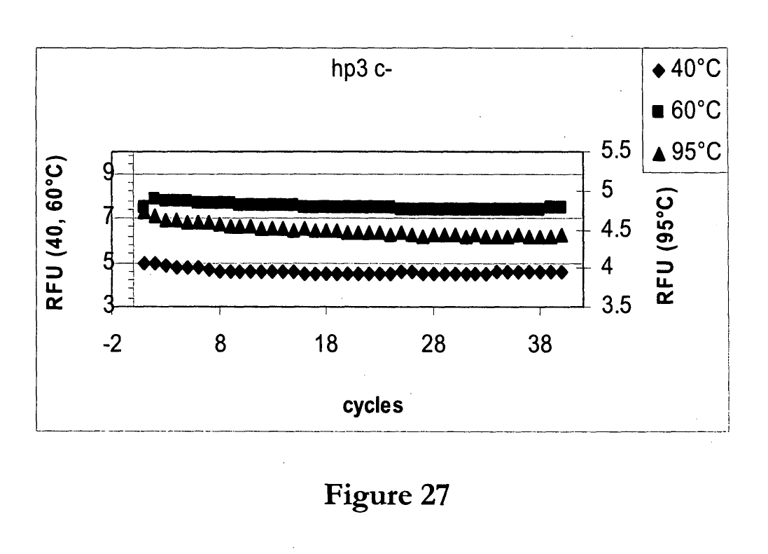

FIG. 27 shows the fluorescence profiles measured at 40, 60 and 95° C., for the negative control for the hp3 “molecular beacon”, as described in example 5.

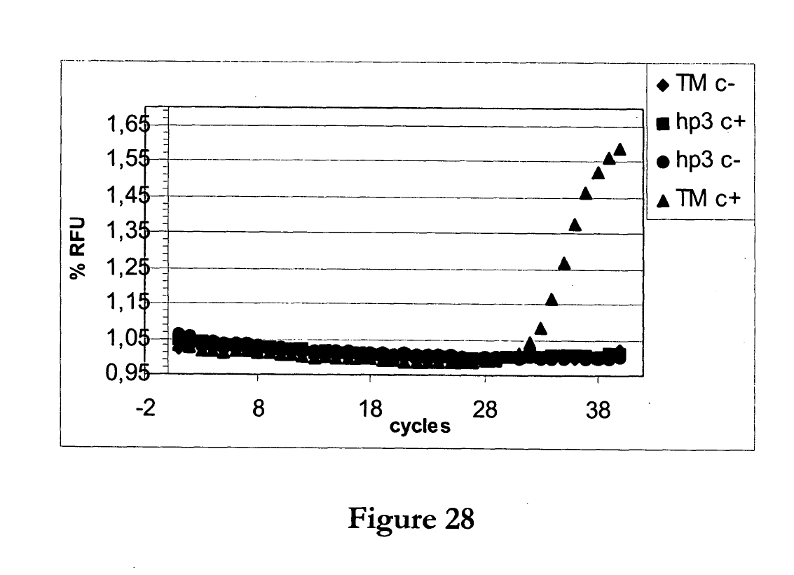

FIG. 28 shows the normalized fluorescence profiles measured at 95° C., for the hp3 “molecular beacon” and the TaqMan probe TM during the amplification on a Lightcycler, as described in example 5.

The following examples are given by way of illustration and are in no way limiting in nature. They will make it possible to understand the invention more clearly.
1. Example of protection, through an arborescence nucleotide structure, against cleavage by the 5′-nuclease activity of a probe acting by molecular recognition

The objective of this experiment was to demonstrate that a probe 5′-modified with an arborescence nucleotide structure is not cleaved by the 5′-nuclease activity of Taq polymerase in an enzyme amplification reaction in vitro.

### 1.a: Definition of the Target, Probes and Primers

The target was the plasmid pCITE with an insert of 1.2 Kb corresponding to a genetic sequence of the hMPV virus. 5×10³ copies per tube.

The sequences used were the following:
sense primer: SEQ ID NO 1: 5′-CAT ATA AGC ATG CTA TAT TAA AAG AGT CTC-3′
reverse primer: SEQ ID NO 2: 5′-CCT ATT TCT GCA GCA TAT TTG TAA TCA G-3′
“molecular beacon” probe (G8): SEQ ID NO 3: 5′-G₈D₄-D₂-D-(dT-FAM)-CGA TGC AAC TGC AGT GAC ACC CTC ATC ATT GCA CAT CG (dT-Dabcyl)-3′
TaqMan probe (TM): SEQ ID NO 4: 5′-FAM-TGC AAT GAT GAG GGT GTC ACT GCG GTT-TAMRA-3′—this sequence specifically recognized a sequence of the amplicon generated in the in vitro amplification.

The underlined part of this sequence specifically recognized a sequence of the amplicon generated in the in vitro amplification.

### 1.b: Synthesis of Probes and Primers

The sequences of 5′-blocked probes and of the primers were synthesized on an Expedite apparatus (Perseptive Biosystems) according to the phosphoramidite method, automatically, in accordance with the protocol proposed by the constructor. The phosphoramidite reagent necessary for the introduction of the “D” unit is a commercially available reagent (ref. 10-1920, Glen Research, USA). This molecule had two reactivated endings, which made it possible to simultaneously couple, in the same synthesis cycle, two nucleotides (Gs in this example) instead of one nucleotide per cycle in the case of the standard reagents. By integrating this molecule three times in the synthesis, a 5′ ending which had eight nucleotides in parallel was created. This arborescence structure was more hindering than a natural ending, and prevented the polymerase from recognizing the 5′ end and from cleaving the “molecular beacon”.

dT-FAM is a modified nucleotide which carried a fluorescein molecule covalently attached to the T base (ref. 10-1056, Glen Research, USA).

dT-Dabcyl was a modified nucleotide which carried a Dabcyl molecule covalently attached to the T base (ref. 10-1058, Glen Research, USA).

The probes and primers are purified by reverse-phase HPLC, their purity is verified by capillary electrophoresis.

The FAM molecule is a fluorescein type fluorophore, the fluorescence of which is detected at 530 nm. The Dabcyl molecule is an aromatic molecule which prevents the emission of fluorescence when it is physically in proximity to the FAM fluorophore.

The TAMRA molecule (derived from tetramethylrhodamine) is an aromatic molecule which prevents the emission of fluorescence from fluorescein by the FRET effect when these two molecules are located in proximity, for example on the same nucleotide sequence. The FRET effect is proportional to the sixth power of the distance between the two molecules.

1.c: PCR Amplification with Real-Time Detection on a LightCycler:

In this example, a “LightCycler FastStart DNA Master Hybridization Probes” amplification kit (Roche, Penzberg, Germany) was used. The preparation of the reaction mixture was carried out according to the procedures recommended by the supplier.

In a reaction volume of 20 μl, 5×10³ copies of the plasmid were mixed with the sense and reverse primers (0.5 μM), the “molecular beacon” (1 μM), 2 μl of vial 1 of the kit (enzyme mix), 0.8 μl of 25 mM MgCl₂ from the kit and PCR grade water from the kit. The reaction mixture was subsequently introduced into a capillary tube and the latter was introduced into the LightCycler. For each amplification reaction, a control was carried out with a TaqMan (TM) probe in place of the “molecular beacon” according to the invention (G8). For each amplification reaction, a control was carried out with the addition of PCR grade water from the kit in place of the target plasmid (negative control, “c−”).

The PCR reaction consisted of an initial denaturation of 8 minutes at 95° C., followed by 40 cycles at 95° C. for 30 seconds, 40° C. for 5 seconds and 60° C. for 60 seconds. The fluorescence was read at 530 nm, at one point at the end of each step in each cycle.

The fluorescence results were analyzed so as to separate the results by temperature, to normalize them and to produce a graphic representation. The graphs obtained for the readings at 40° C. and 60° C. serve to verify the hybridization of the probe with the amplicons during the amplification reaction. At 95° C., on the other hand, there is no hybridization possible between the probe and the amplicons. Any increase in signal at 95° C., compared with the negative control, could only be due to prior cleavage of the probe. The graph obtained for the reading at 95° C. therefore serves to prove probe cleavage or the absence of probe cleavage by the 5′-nuclease activity of the Taq polymerase enzyme (or TaqPol).

### 1.d: Results and Discussion:

The profiles obtained with the TaqMan probe TM in real-time detection are shown in FIGS. 3 and 4.

The horizontal axis represents the number of amplification cycles, the vertical axis represents the fluorescence detected at 530 nm, at 40, 60° C. and 95° C., in each cycle. The profiles at 60 and 40° C. demonstrate that the TaqMan probe TM specifically detects the presence of the amplicons derived from the in vitro enzyme amplification reaction. The profiles at 95° C. demonstrate that the TM probe is specifically cleaved during the amplification reaction.

The profiles obtained with the G8 “molecular beacon” in real-time detection are shown in FIGS. 5 and 6, in which the horizontal axis represents the number of amplification cycles, the left vertical axis represents the fluorescence detected at 530 nm, at 40 and 60° C., in each cycle; and the right vertical axis represents the fluorescence detected at 530 nm, at 95° C., in each cycle.

The profiles at 60 and 40° C. demonstrate that the G8 “molecular beacon” specifically detected the presence of the amplicons derived from the in vitro enzyme amplification reaction. The profiles at 95° C. demonstrate that the G8 “molecular beacon” was not cleaved under the conditions under which the TaqMan probe TM is cleaved.

FIG. 7 shows the comparison of the increases in fluorescence at 95° C. for the TaqMan probe TM and for the G8 modified “molecular beacon”. A specific increase in the fluorescence could be observed only with the TM probe when the amplification takes place. When the amplification does not take place (negative controls) and when the “molecular beacon” is modified, G8, there is no cleavage by the 5′-nuclease activity of the Taq polymerase, and there is therefore no increase in fluorescence.

Conclusion: The G8 “molecular beacon” according to the invention hybridizes specifically with the amplicons derived from the cycling on a lightcycler, producing a real-time detection comparable to that obtained with the TaqMan probe TM. The G8 “molecular beacon” is not a substrate for the cleavage by the 5′-nuclease activity of Taq polymerase, unlike the TaqMan probe TM.
2. Protection against cleavage by the 5′-nuclease activity with a nucleotide sequence forming a thermostable hairpin structure of a probe acting by molecular recognition (“molecular beacon”)

The objective of this experiment was to demonstrate that a “molecular beacon” modified in its 5′ side with a nucleotide sequence forming a thermostable hairpin was not cleaved by the 5′-nuclease activity of Taq polymerase in an in vitro enzyme amplification reaction.

### 2.a: Definition of the Target, Probes and Primers

The target is the plasmid pCITE with an insert of 1.2 Kb corresponding to a genetic sequence of the hMPV virus (5×10³ copies per tube).
sense primer: SEQ ID NO 5: 5′-CAT ATA AGC ATG CTA TAT TAA AAG AGT CTC-3′
reverse primer: SEQ ID NO 6: 5′-CCT ATT TCT GCA GCA TAT TTG TAA TCA G-3′
“molecular beacon” (hp5): SEQ ID NO 7: 5′-AT GCA TCG TTT TTC GAT GC AT(dT-FAM) CGA TGC AAC TGC AGT GAC ACC CTC ATC ATT GCA CAT CG (dT-Dabcyl)-3′
TaqMan probe (TM): SEQ ID NO 8: 5′-FAM-TGC AAT GAT GAG GGT GTC ACT GCG GTT-TAMRA-3′—this sequence specifically recognized a sequence of the amplicon generated in the in vitro amplification.

The underlined part of this sequence specifically recognized a sequence of the amplicon generated in the in vitro amplification. The part of this sequence between the 5′ end and the fluorophore forms a stable hairpin structure.

### 2.b: Synthesis of Probes and Primers

The sequences of probes and of the primers were synthesized according to the protocol described in example 1. b.

dT-Dabcyl is a modified nucleotide which carries a Dabcyl molecule covalently attached to the T base (ref. 10-1058, Glen Research, USA). dT-FAM is a modified nucleotide which carries a fluorescein molecule covalently attached to the T base (ref 10-1056, Glen Research, USA).

FAM is a fluorescein-type fluorophore, the fluorescence of which can be detected at 530 nm.

Dabcyl is an aromatic molecule which prevents the emission of fluorescence when it is physically in proximity to a fluorophore, it is therefore a “quencher”.

The TAMRA molecule (tetramethylrhodamine derivative) is an aromatic molecule which prevents the emission of fluorescence from fluorescein through the FRET effect when these two molecules are located in proximity, for example on the same nucleotide sequence. The FRET effect is proportional to the sixth power of the distance between the two molecules.

2.c. PCR Amplification with Real-Time Detection on a LightCycler:

In this example, a “LightCycler FastStart DNA Master Hybridization Probes” amplification kit (Roche, Penzberg, Germany) was used. The preparation of the reaction mixture was carried out according to the procedures recommended by the supplier. In a reaction volume of 20 μl, 5×10³ copies of the plasmid were mixed with the sense and reverse primers (0.5 μM), the hp5 modified “molecular beacon” (1 μM), 2 μl of vial 1 from the kit (enzyme mix), 0.8 μl of 25 mM MgCl₂ from the kit and PCR grade water from the kit.

The reaction mixture was subsequently introduced into a capillary tube and the latter was introduced into the LightCycler.

For each amplification reaction, a control was carried out with the TaqMan probe (TM) in place of the hp5 modified “molecular beacon”.

For each amplification reaction, a control was carried out with the addition of PCR grade water from the kit in place of the target plasmid (negative control, “c−”).

The PCR reaction consisted of an initial denaturation of 8 minutes at 95° C., followed by 40 cycles at 95° C. for 30 seconds, 40° C. for 5 seconds and 60° C. for 60 seconds.

The fluorescence was read at 530 nm, at one point at the end of each step in each cycle. The fluorescence results were subsequently analyzed so as to separate the results by temperature, to normalize them and to produce a graphic representation. The graphs obtained for the readings at 40° C. and 60° C. serve to verify the hybridization of the probe with the amplicons during the amplification reaction. At 95° C., on the other hand, there was no hybridization possible between the probe and the amplicons. Any increase in signal at 95° C., compared with the negative control, could only be due to cleavage of the probe. The graph obtained for the reading at 95° C. therefore serves to prove whether or not the probe is cleaved by the 5′-nuclease activity of TagPol.

2.d. Results and Discussion

The profiles obtained in real-time detection are shown in FIGS. 8 to 12, in which the horizontal axis represents the number of amplification cycles, the left vertical axis (FIGS. 8 to 11) represents the fluorescence detected at 530 nm, at 40 and 60° C., in each cycle, and the right vertical axis (left in FIG. 12) represents the fluorescence detected at 530 nm, at 95° C., in each cycle.

The profiles at 60 and 40° C. demonstrate that the hp5 “molecular beacon” according to the invention, and also the TM probe, specifically detected the presence of the amplicons derived from the in vitro enzyme amplification reaction.

The profiles at 95° C. demonstrated that the TM probe was specifically cleaved during the amplification reaction, whereas hp5 was not cleaved under the same conditions.

FIG. 12 shows the comparison of the increases in fluorescence at 95° C. for the hp5 modified “molecular beacon” and for the TM probe.

A specific increase in the fluorescence could be observed only when the TM probe was used when the amplification took place. When the amplification did not take place (negative controls) and when the modified “molecular beacon” was used, there was no cleavage by the 5′-nuclease activity of the Taq polymerase, and there was therefore no increase in fluorescence.

Conclusion: The hp5 modified “molecular beacon” specifically hybridized with the amplicons derived from the cycling on a lightcycler, producing a real-time detection comparable to that obtained with the TaqMan probe TM. The hp5 modified “molecular beacon” was not cleaved by the 5′-nuclease activity of Taq polymerase, unlike the TaqMan probe.
3. Protection against cleavage by the 5′-nuclease activity with a nucleotide sequence forming a thermostable hairpin structure of a probe acting by molecular recognition (“molecular beacon”)

The objective of this experiment was to demonstrate that a “molecular beacon” modified in its 5′ side with a nucleotide sequence forming a thermostable hairpin was not cleaved by the 5′-nuclease activity of Taq polymerase in an in vitro enzyme amplification reaction.

Compared with the previous example, the “molecular beacon” has a psoralene molecule at its 5′ end. This molecule serves to render the hairpin more thermostable compared with the “molecular beacon” without psoralene. This molecule intercalates into the hairpin structure to which it is attached and stabilizes this structure, allowing it to remain stable at the polymerization temperature.

### 3.a: Definition of the Target, Probes and Primers

The target is the plasmid pCITE with an insert of 1.2 Kb corresponding to a genetic sequence of the hMPV virus. 5×10³ copies per tube.

The sequences used in this example were the following:
sense primer: SEQ ID NO 5: 5′-CAT ATA AGC ATG CTA TAT TAA AAG AGT CTC-3′
reverse primer: SEQ ID NO 6: 5′-CCT ATT TCT GCA GCA TAT TTG TAA TCA G-3′

“molecular beacon” (hp2): SEQ ID NO 9: 5′-Pso-GCA TCG TTT TTC GAT GC(dT-FAM)-T CGA TGC AAC TGC AGT GAC ACC CTC ATC ATT GCA CAT CG (dT-Dabcyl)-3′
control “molecular beacon” (MBnm): SEQ ID NO 10: 5′-(dT-FAM)-CGA TGC AAC TGC AGT GAC ACC CTC ATC ATT GCA CAT CG (dT-Dabcyl)-3′—this sequence specifically recognized a sequence of the amplicon generated in the in vitro amplification. The underlined part of this sequence specifically recognized a sequence of the amplicon generated in the in vitro amplification.

### 3.b: Synthesis of Probes and Primers

The sequences of probes and of the primers were synthesized according to the protocol described in example 1. b.

“Pso” is a psoralene molecule which is integrated into the sequence during the automatic synthesis using commercially available phosphoramidite (ref. 10-1982, Glen Research, USA). This molecule intercalates into the hairpin structure to which it is attached, and stabilizes this structure, allowing it to remain stable at the polymerization temperature.

dT-FAM is a modified nucleotide which carries a fluorescein molecule covalently attached to the T base (ref 10-1056, Glen Research, USA).

dT-Dabcyl is a modified nucleotide which carries a Dabcyl molecule covalently attached to the T base (ref. 10-1058, Glen Research, USA).

FAM is a fluorescein-type fluorophore, the fluorescence of which can be detected at 530 nm.

Dabcyl is an aromatic molecule which prevents the emission of fluorescence when it is physically in proximity to the FAM fluorophore.

3.c. PCR Amplification with Real-Time Detection on a LightCycler:

In this example, a “LightCycler FastStart DNA Master Hybridization Probes” amplification kit (Roche, Penzberg, Germany) was used. The preparation of the reaction mixture was carried out according to the procedures recommended by the supplier. In a reaction volume of 20 μl, 5×10³ copies of the plasmid were mixed with the sense and reverse primers (0.5 μM), the hp2 “molecular beacon” according to the invention (1 μM), 2 μl of vial 1 from the kit (enzyme mix), 0.8 μl of 25 mM MgCl₂ from the kit and PCR grade water from the kit.

The reaction mixture was subsequently introduced into a capillary tube and the latter was introduced into the LightCycler.

For each amplification reaction, a control was carried out with the control “molecular beacon” (MBnm) in place of the hp2 “molecular beacon” according to the invention.

For each amplification reaction, a control was carried out with the addition of PCR grade water from the kit in place of the target plasmid (negative control, “c−”).

The PCR reaction consisted of an initial denaturation of 8 minutes at 95° C., followed by 40 cycles at 95° C. for 30 seconds, 40° C. for 5 seconds and 60° C. for 60 seconds.

The fluorescence was read at 530 nm, at one point at the end of each step in each cycle. The fluorescence results were subsequently analyzed so as to separate the results by temperature, to normalize them and to produce a graphic representation. The graphs obtained for the readings at 40° C. and 60° C. serve to verify the hybridization of the probe with the amplicons during the amplification reaction. At 95° C., on the other hand, there was no hybridization possible between the probe and the amplicons. Any increase in signal at 95° C., compared with a negative control, could only be due to cleavage of the probe. The graph obtained for the reading at 95° C. therefore serves to prove whether or not the probe is cleaved by the 5′-nuclease activity of TaqPol.

3.d. Results and Discussion

The profiles obtained in real-time detection are shown in FIGS. 13 to 17, in which the horizontal axis represents the number of amplification cycles, the left vertical axis (FIGS. 13 to 16) represents the fluorescence detected at 530 nm, at 40 and 60° C., in each cycle, and the right vertical axis (left in FIG. 17) represents the fluorescence detected at 530 nm, at 95° C., in each cycle.

The profiles at 60 and 40° C. demonstrate that the hp2 “molecular beacon” according to the invention and the MBnm control specifically detected the presence of the amplicons derived from the in vitro enzyme amplification reaction.

The profiles at 95° C. demonstrated that MBnm was specifically cleaved during the amplification reaction, whereas hp2 was not cleaved under the same conditions.

FIG. 17 shows the comparison of the increases in fluorescence at 95° C. for the unmodified “molecular beacon” MBnm and for the modified “molecular beacon” hp2.

A specific increase in the fluorescence could be observed only when the MBnm “molecular beacon” was used when the amplification takes place. When the amplification does not take place (negative controls) and when the “molecular beacon” is modified according to the invention, there was no cleavage by the 5′-nuclease activity of Taq polymerase, and there was therefore no increase in fluorescence.

Conclusion: The hp2 modified “molecular beacon” hybridized specifically with the amplicons derived from the cycling on a lightcycler, producing a real-time detection comparable to that obtained with the MBnm unmodified “molecular beacon”. The hp2 modified “molecular beacon” was not cleaved by the 5′-nuclease activity of Taq polymerase, unlike the MBnm unmodified “molecular beacon”.
4. Example of protection against cleavage by the 5′-nuclease activity with a nucleotide sequence forming a thermostable hairpin structure of a probe acting by molecular recognition (“molecular beacon”)

The objective of this experiment is to demonstrate that a “molecular beacon” modified in its 5′ side with a nucleotide sequence forming a thermostable hairpin is not a substrate for cleavage by the 5′-nuclease activity of Taq polymerase in an in vitro enzyme amplification reaction.

Compared with examples 2 and 3, this modified molecular beacon comprises a fluorophore in its 5′ end, within the hairpin structure, whereas in the previous cases, the fluorescein was located in the first segment of the “molecular beacon” (see FIG. 2, right).

### 4.a: Definition of the Target, Probes and Primers

The target is the plasmid pCITE with an insert of 1.2 Kb corresponding to a genetic sequence of the hMPV virus (5×10³ copies per tube).
sense primer: SEQ ID NO 5: 5′-CAT ATA AGC ATG CTA TAT TAA AAG AGT CTC-3′
reverse primer: SEQ ID NO 6: 5′-CCT ATT TCT GCA GCA TAT TTG TAA TCA G-3′
“molecular beacon” (hp4): SEQ ID NO 9: 5′-FAM-GCA TCG TTT TTC GAT GC CGA TGC AAC TGC AGT GAC ACC CTC ATC ATT GCA CAT CG (dT-Dabcyl)-3′
TaqMan probe (TM): SEQ ID NO 8: 5′-FAM-TGC AAT GAT GAG GGT GTC ACT GCG GTT-TAMRA-3′—this sequence specifically recognized a sequence of the amplicon generated in the in vitro amplification.

The underlined part of this sequence specifically recognized a sequence of the amplicon generated in the in vitro amplification.

### 4.b: Synthesis of Probes and Primers

The sequences of probes and of the primers were synthesized according to the protocol described in example 1. b.

dT-Dabcyl is a modified nucleotide which carries a Dabcyl molecule covalently attached to the T base (ref. 10-1058, Glen Research, USA).

dT-FAM is a modified nucleotide which carries a fluorescein molecule covalently attached to the T base (ref 10-1056, Glen Research, USA).

FAM is a fluorescein-type fluorophore, the fluorescence of which can be detected at 530 nm.

Dabcyl is an aromatic molecule which prevents the emission of fluorescence when it is physically in proximity to a fluorophore, it is therefore a “quencher”.

The TAMRA molecule (tetramethylrhodamine derivative) is an aromatic molecule which prevents the emission of fluorescence from fluorescein through the FRET effect when these two molecules are located in proximity, for example on the same nucleotide sequence. The FRET effect is proportional to the sixth power of the distance between the two molecules.

4.c. PCR Amplification with Real-Time Detection on a LightCycler:

In this example, a “LightCycler FastStart DNA Master Hybridization Probes” amplification kit (Roche, Penzberg, Germany) was used. The preparation of the reaction mixture was carried out according to the procedures recommended by the supplier. In a reaction volume of 20 μl, 5×10³ copies of the plasmid were mixed with the sense and reverse primers (0.5 μM), the hp4 “molecular beacon” (1 μM), 2 μl of vial 1 from the kit (enzyme mix), 0.8 μl of 25 mM MgCl₂ from the kit and PCR grade water from the kit.

The reaction mixture was subsequently introduced into a capillary tube and the latter was introduced into the LightCycler.

For each amplification reaction, a control was carried out with the TaqMan probe (TM) in place of the hp4 modified “molecular beacon”.

For each amplification reaction, a control was carried out with the addition of PCR grade water from the kit in place of the target plasmid (negative control, “c−”).

The PCR reaction consisted of an initial denaturation of 8 minutes at 95° C., followed by 40 cycles at 95° C. for 30 seconds, 40° C. for 5 seconds and 60° C. for 60 seconds.

The fluorescence was read at 530 nm, at one point at the end of each step in each cycle. The fluorescence results were subsequently analyzed so as to separate the results by temperature, to normalize them and to produce a graphic representation. The graphs obtained for the readings at 40° C. and 60° C. serve to verify the hybridization of the probe with the amplicons during the amplification reaction. At 95° C., on the other hand, there was no hybridization possible between the probe and the amplicons. Any increase in signal at 95° C., compared with the negative control, could only be due to cleavage of the probe. The graph obtained for the reading at 95° C. therefore serves to prove whether or not the probe is cleaved by the 5′-nuclease activity of TaqPol.

4.d. Results and Discussion

The profiles obtained in real-time detection are shown in FIGS. 18 to 22, in which the horizontal axis represents the number of amplification cycles, the left vertical axis (FIGS. 18 to 21) represents the fluorescence detected at 530 nm, at 40 and 60° C., in each cycle, and the right vertical axis (left in FIG. 22) represents the fluorescence detected at 530 nm, at 95° C., in each cycle.

The profiles at 60 and 40° C. demonstrate that the hp4 modified “molecular beacon” and also the TM probe specifically detected the presence of the amplicons derived from the in vitro enzyme amplification reaction.

The profiles at 95° C. demonstrated that the TM probe was specifically cleaved during the amplification reaction, whereas hp4 was not cleaved under the same conditions.

FIG. 22 represents the comparison of the increases in fluorescence at 95° C. for the hp4 modified “molecular beacon” and for the TM probe.

A specific increase in the fluorescence could only be observed when the TM probe was used when the amplification takes place. When the amplification does not take place (negative controls) and when the modified “molecular beacon” was used, there was no cleavage by the 5′-nuclease activity of Taq polymerase and there was therefore no increase in fluorescence.

Conclusion: The hp4 “molecular beacon” according to the invention specifically hybridized with the amplicons derived from the cycling on a lightcycler, producing a real-time detection comparable to that obtained with the TaqMan probe TM. The hp4 “molecular beacon” modified according to the invention was not cleaved by the 5′-nuclease activity of Taq polymerase, unlike the TaqMan probe.
5. Example of protection against cleavage by the 5′-nuclease activity with a nucleotide sequence forming a thermostable hairpin structure of a probe acting by molecular recognition (“molecular beacon”)

The objective of this experiment is to demonstrate that a “molecular beacon” modified in its 5′ side with a nucleotide sequence forming a thermostable hairpin is not a substrate for cleavage by the 5′-nuclease activity of Taq polymerase in an in vitro enzyme amplification reaction.

Compared with examples 2 and 3, this modified molecular beacon comprises a fluorophore in its 5′ end, within the hairpin structure, whereas in examples 2 and 3, the fluorescein was located in the first fragment of the “molecular beacon” (see FIG. 2, right).

Compared with example 4, this modified “molecular beacon” comprises a nucleotide T between the first fragment of the “molecular beacon” and the hairpin structure. This nucleotide distances the double helix formed by the hairpin structure from that formed by the first and third fragments of the “molecular beacon”, thereby decreasing the interactions with one another.

### 5.a: Definition of the Target, Probes and Primers

The target is the plasmid pCITE with an insert of 1.2 Kb corresponding to a genetic sequence of the hMPV virus (5×10³ copies per tube).
sense primer: SEQ ID NO 5: 5′-CAT ATA AGC ATG CTA TAT TAA AAG AGT CTC-3′
reverse primer: SEQ ID NO 6: 5′-CCT ATT TCT GCA GCA TAT TTG TAA TCA G-3′
“molecular beacon” (hp3): SEQ ID NO 10: 5′-FAM-GCA TCG TTT TTC GAT GCT CGA TGC AAC TGC AGT GAC ACC CTC ATC ATT GCA CAT CG (dT-Dabcyl)-3′
TaqMan probe (TM): SEQ ID NO 8: 5′-FAM-TGC AAT GAT GAG GGT GTC ACT GCG GTT-TAMRA-3′—this sequence specifically recognized a sequence of the amplicon generated in the in vitro amplification.

The underlined part of this sequence specifically recognized a sequence of the amplicon generated in the in vitro amplification.

### 5.b: Synthesis of Probes and Primers

The sequences of probes and of the primers were synthesized according to the protocol described in example 1. b.

dT-Dabcyl is a modified nucleotide which carries a Dabcyl molecule covalently attached to the T base (ref. 10-1058, Glen Research, USA).

dT-FAM is a modified nucleotide which carries a fluorescein molecule covalently attached to the T base (ref 10-1056, Glen Research, USA).

FAM is a fluorescein-type fluorophore, the fluorescence of which can be detected at 530 nm.

Dabcyl is an aromatic molecule which prevents the emission of fluorescence when it is physically in proximity to a fluorophore, it is therefore a “quencher”.

The TAMRA molecule (tetramethylrhodamine derivative) is an aromatic molecule which prevents the emission of fluorescence from fluorescein through the FRET effect when these two molecules are located in proximity, for example on the same nucleotide sequence. The FRET effect is proportional to the sixth power of the distance between the two molecules.

5.c. PCR Amplification with Real-Time Detection on a LightCycler:

In this example, a “LightCycler FastStart DNA Master Hybridization Probes” amplification kit (Roche, Penzberg, Germany) was used. The preparation of the reaction mixture was carried out according to the procedures recommended by the supplier. In a reaction volume of 20 μl, 5×10³ copies of the plasmid were mixed with the sense and reverse primers (0.5 μM), the hp3 “molecular beacon” (1 μM), 2 μl of vial 1 from the kit (enzyme mix), 0.8 μl of 25 mM MgCl₂ from the kit and PCR grade water from the kit.

The reaction mixture was subsequently introduced into a capillary tube and the latter was introduced into the LightCycler.

For each amplification reaction, a control was carried out with the TaqMan probe (TM) in place of the hp3 “molecular beacon” according to the invention.

For each amplification reaction, a control was carried out with the addition of PCR grade water from the kit in place of the target plasmid (negative control, “c−”).

The PCR reaction consisted of an initial denaturation of 8 minutes at 95° C., followed by 40 cycles at 95° C. for 30 seconds, 40° C. for 5 seconds and 60° C. for 60 seconds.

The fluorescence was read at 530 nm, at one point at the end of each step in each cycle. The fluorescence results were subsequently analyzed so as to separate the results by temperature, to normalize them and to produce a graphic representation. The graphs obtained for the readings at 40° C. and 60° C. serve to verify the hybridization of the probe with the amplicons during the amplification reaction. At 95° C., on the other hand, there was no hybridization possible between the probe and the amplicons. Any increase in signal at 95° C., compared with the negative control, could only be due to cleavage of the probe. The graph obtained for the reading at 95° C. therefore serves to prove whether or not the probe is cleaved by the 5′-nuclease activity of TaqPol.

The profiles obtained in real-time detection are shown in FIGS. 23 to 27, in which the horizontal axis represents the number of amplification cycles, the left vertical axis (FIGS. 23 to 26) represents the fluorescence detected at 530 nm, at 40 and 60° C., in each cycle, and the right vertical axis (left in FIG. 27) represents the fluorescence detected at 530 nm, at 95° C., in each cycle.

The profiles at 60 and 40° C. demonstrate that the hp3 modified “molecular beacon”, and also the TM probe, specifically detected the presence of the amplicons derived from the in vitro enzyme amplification reaction.

The profiles at 95° C. demonstrated that the TM probe was specifically cleaved during the amplification reaction, whereas hp3 was not cleaved under the same conditions.

FIG. 27 shows the comparison of the increases in fluorescence at 95° C. for the hp3 modified “molecular beacon” and for the TM probe.

A specific increase in the fluorescence could only be observed when the TM probe is used when the amplification takes place. When the amplification does not take place (negative controls) and when the modified “molecular beacon” is used, there was no cleavage by the 5′-nuclease activity of Taq polymerase, and there was therefore no increase in fluorescence.
a) Conclusion: The hp3 modified “molecular beacon” specifically hybridized with the amplicons derived from the cycling on a lightcycler, producing a real-time detection comparable to that obtained with the TaqMan probe TM. The hp3 modified “molecular beacon” was not cleaved by the 5′-nuclease activity of Taq polymerase,

1. A method for blocking a 5′ nuclease activity of a thermostable polymerase enzyme during a polymerase chain reaction utilizing at least one detection probe acting by molecular recognition, the detection probe comprising successively in the 5′ 33′ direction:
a first nucleotide segment comprising a sterically hindering hairpin structure at its 5′ end, and
a second nucleotide segment, complementary to the target sequence.

2. The method of claim 1, wherein the detection probe also comprises, downstream of said second nucleotide segment, a third nucleotide segment capable of hybridizing to said first nucleotide segment.

3. The method of claim 1, wherein the detection probe is labeled at one end with a fluorophore and at the other end with a quencher.

4. The method of claim 3, wherein said fluorophore is fluorescein.

5. The method of claim 1, wherein said first nucleotide segment comprises from 3 to 8 nucleotides.

6. The method of claim 1, wherein said second nucleotide segment comprises from 10 to 35 nucleotides.

7. The method of claim 2, wherein said third segment comprises from 3 to 8 nucleotides.
